(19) United States
(12) Patent Application Publication
(10) Pub. No.: US 2023/0346666 A1
(43) Pub. Date: Nov. 2, 2023

(54) Personal Care Composition

(71) Applicant: Colgate-Palmolive Company, New York, NY (US)

(72) Inventors: Vinay Bhardwaj, Piscataway, NJ (US); Junhong Mao, Plainsboro, NJ (US); Thomas Boyd, Metuchen, NJ (US); Ewelina Lesniak, Linden, NJ (US); Savitha Rajagopal, Chandler, AZ (US)

(73) Assignee: Colgate-Palmolive Company, New York, NY (US)

(21) Appl. No.: 18/305,746

(22) Filed: Apr. 24, 2023

Related U.S. Application Data
(60) Provisional application No. 63/336,703, filed on Apr. 29, 2022.

(51) Int. Cl. A61K 8/44; A61K 8/365; A61Q 19/02; A61Q 19/08

(52) U.S. Cl. CPC A61K 8/44; A61K 8/365; A61Q 19/02; A61Q 19/08

(57) ABSTRACT

Personal care compositions and, particularly, personal care compositions for depigmenting keratin materials are disclosed herein. The personal care compositions typically include tranexamic acid and mandelic acid, wherein the personal care composition has a weight ratio of tranexamic acid to mandelic acid of from about 1:6 to about 1:17.

### CROSS-REFERENCE TO RELATED APPLICATION

This application claims the benefit of priority from U.S. Provisional Application No. 63/336,703, filed Apr. 29, 2022 and titled “PERSONAL CARE COMPOSITIONS,” the contents of which are hereby incorporated herein in their entireties, for all purposes.

### BACKGROUND

The color of human skin is mainly determined by the nature and concentration of a pigment, melanin. There are two types of melanin in epidermal cells, eumalanin, which is a brown-black color pigment, and pheomelanin, which is a yellow-orange color pigment. Melanin is synthesized by specific dendritic cells, called melanocytes, located in the basal layer of the epidermis. Melanogenesis, i.e. the formation of melanin, takes place in specific organelles, the melanosomes, which, loaded with melanin, are transferred to neighboring epidermal cells, the keratinocytes, via the dendrites.

The mechanism of melanogenesis is complex and schematically involves the following main steps: Tyrosine to Dopa, Dopa to Dopaquinone, Dopaquinone to Dopachrome, and Dopachrome to Melanin. Inside the melanosomes, three enzymes, namely tyrosinase, Tyrp 1 (for Tyrosinase-related protein 1) and Dct/Tyrp2 (for DOPAchrome tautomerase/Tyrosinase-related protein 2), play a major role in melanin production. More particularly, tyrosinase is an essential enzyme that regulates early steps of eumelanin and pheomelanin synthesis. The pigmentation of skin on the face and/or of the body depends on various factors, such as environmental factors linked to the seasons of the year, and the individual's origin.

Additionally, at various periods of one's life, darker and/or more highly colored spots may form, which give the skin heterogeneity. For example, colored spots (also referred to as sunspots) may form on the hands of an individual. These spots are typically due to a high concentration of melanin in the skin. Some individuals do not like the colored spots or heterogeneity of their skin coloring and, thus, there remains a need for depigmentation products for skin.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with one aspect of the invention, provided is a personal care composition that includes tranexamic acid and mandelic acid, wherein the personal care composition has a weight ratio of tranexamic acid to mandelic acid of from about 1:6 to about 1:17. In some embodiments, the personal care compositions are formulated to have a weight ratio of tranexamic acid to mandelic acid from about 1:7 to about 1:12.

According to another aspect, provided is a personal care composition having from about 0.1 to about 10 wt. % of tranexamic acid; from about 2 to about 20 wt. % of mandelic acid; from about 0.5 to about 10 wt. % of sodium pyruvate; and optionally, from about 1 to about 30 wt. % of one or more additional acid(s) selected from lactic acid, maleic acid, succinic acid, formic acid, propionic acid, butyric acid, isobutyric acid, 2-methylpropanoic acid, valeric acid, a derivative thereof, and a combination of two or more thereof, wherein all weight percentages are based on the total weight of the personal care composition. The personal care composition typically has a weight ratio of tranexamic acid to mandelic acid from about 1:6 to about 1:17.

### BRIEF DESCRIPTION OF THE DRAWINGS

Implementation of the present technology will now be described, by way of example only, with reference to the attached figures, wherein:

FIG. 1 is a bar graph of melanin reduction in skin explants seven days after a single application of one of two comparative compositions or a non-limiting, exemplary composition according to aspects of the invention;

FIG. 2 is a bar graph of the collagen produced in skin explants seven days after a single application of one of two comparative compositions or a non-limiting, exemplary composition according to aspects of the invention;

FIG. 3 is a bar graph of the mRNA levels for TYR, TYRP1, and DCT in skin explants seven days after a single application of one of two comparative compositions or a non-limiting, exemplary composition according to aspects of the invention;

FIG. 4 is a bar graph of the binding affinity of compositions containing tranexamic acid in combination with mandelic acid, lactic acid, or pyruvic according to aspects of the invention;

FIG. 5 is a graph of the binding affinity of certain compounds to tyrosinase according to aspects of the invention;

FIG. 6A is an image of dermoscope and vivascope images revealing a reduction of acne-induced PIH spots and the complete closure of three comedones after application of a non-limiting, exemplary composition according to aspects of the invention;

FIG. 6B is a bar graph of the average score calculated from the dermatologist's objective visual assessment of each subject before and after chemical peeling a non-limiting, exemplary composition according to aspects of the invention;

FIGS. 7A-7D are graphs of the microbiome and changes in the microbiome after application of a non-limiting, exemplary composition according to aspects of the invention; and

FIG. 8 is a bar graph showing the synergistic effect achieved by the combination of mandelic acid, lactic acid, and pyruvic acid according to aspects of the invention.

It should be understood that the various aspects are not limited to the compositions, compounds, and instrumentality shown in the drawings.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but not limited to”. The term “including” should be interpreted as “including, but not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “μL” refers to a microliter, or 10⁻⁶ liters. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

The phrase “MRS agar” refers to De Man, Rogosa and Sharpe agar, which is a selective culture medium designed to favor the growth of Lactobacillus. The phrase “TSB medium” refers to tryptic soy broth or trypticase soy broth, which is used in microbiology laboratories as a culture broth to grow aerobic bacteria. The phrase “PBS wash” refers to phosphate-buffered saline wash. The abbreviation “qPCR” is quantitative polymerase chain reaction.

When referring to chemical structures, and names, the symbols “C”, “H”, and “0” mean carbon, hydrogen, and oxygen, respectively. The symbols “—”, “═” and “≡” mean single bond, double bond, and triple bond respectively. For readability purposes, the chemical functional groups are in their adjective form; for each of the adjective, the word “group” is assumed. For example, the adjective “alkyl” without a nouns thereafter, should be read as “an alkyl group.”

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the personal care composition on a dry matter basis.

Some of the various categories of components identified may overlap. In such cases where overlap may exist and the personal care composition includes both components (or the composition includes more than two components that overlap), an overlapping compound does not represent more than one component. For example, certain components or ingredients may be characterized as containing both a pH adjuster and an acid having a carbon group comprising 1 to 6. If a particular personal care composition recites both a pH adjuster and an alpha hydroxyl acid, a compound (e.g., citric acid) that may be characterized as both a pH adjuster and an alpha hydroxyl acid will serve only as either the pH adjuster or the alpha hydroxyl acid—not both.

Aspects of the invention relate to personal care compositions and particularly personal care compositions adapted for reducing skin pigmentation and/or promoting homogeneity of skin pigmentation. The inventors recognized that there remains a need for improved compositions for depigmenting keratin materials, especially the skin, which also makes it possible to improve the homogeneity of the complexion and to revive the radiance of the complexion. The inventors discovered that certain combinations of ingredients in specific weight ratios enable personal care compositions to be formulated that provide enhanced pigment reduction and/or elimination while simultaneously increasing collagen growth.

In accordance with a first aspect of the invention, provided is a personal care composition that includes tranexamic acid and mandelic acid, wherein the personal care composition has a weight ratio of tranexamic acid to mandelic acid of from about 1:6 to about 1:17.

The personal care compositions may be formulated to have a weight ratio of tranexamic acid to mandelic acid of from about 1:6 to about 1:14, about 1:6 to about 1:12, about 1:6 to about 1:10, about 1:6 to about 1:8; from about 1:7 to about 1:14, about 1:7 to about 1:12, about 1:7 to about 1:10, about 1:7 to about 1:8; from about 1:8 to about 1:14, about 1:8 to about 1:12, about 1:8 to about 1:10, about 1:6 to about 1:9; from about 1:9 to about 1:14, about 1:9 to about 1:12, about 1:9 to about 1:10; from about 1:10 to about 1:14, about 1:10 to about 1:12; from about 1:11 to about 1:14, about 1:11 to about 1:12, or any range or subrange thereof.

The inventors discovered that the weight ratio of tranexamic acid to mandelic acid may provide a synergistic and/or an enhanced effect benefit beyond that which was expected. For example, preferable weight ratios of tranexamic acid to mandelic acid provide enhanced pigment reduction as compared to similar amounts and ratios of tranexamic acid and lactic and/or pyruvic acid. Without being limited to any particular theory, the inventors believe that the synergistic and/or enhanced effect benefit achieved by tranexamic acid and mandelic acid may be at least partly be due to tranexamic acid and mandelic acid binding to different sites on important enzyme (e.g., human tyrosinase) for melanin production.

Additionally or alternatively, the personal care compositions can unexpectedly achieve improved effects by being formulated to have lactic acid, mandelic acid, and/or pyruvic acid in certain weight ratios. For example, the personal care compositions may be formulated to have a weight ratio of lactic acid to mandelic acid to pyruvic acid (lactic acid:mandelic acid:pyruvic acid) of from about 12:6:1 to about 1:1:1, about 6:4:1 to about 1:1:1, about 5:3:1 to about 1:1:1, about 4:2:1 to about 1:1:1; from about 12:6:1 to about 2:1.5:1, about 6:4:1 to about 2:1.5:1, about 5:3:1 to about 2:1.5:1, about 4:2:1 to about 2:1.5:1; from about 12:6:1 to about 3:1.75:1, about 6:4:1 to about 3:1.75:1, about 5:3:1 to about 3:1.75:1, about 4:2:1 to about 3:1.75:1, or any range or subrange thereof. Without being limited to any particular theory, it is believed that the combination of at least two of lactic acid, mandelic acid, and pyruvic acid may induce an effect that unexpectedly promotes tranexamic acid penetration across the skin barrier.

In certain embodiments, the personal care composition may be formulated to have a weight ratio of alpha hydroxyl acid (lactic acid and/or mandelic acid) to keto acid (e.g. pyruvic acid) of about 12:1 to about 1:1. For example, the personal care composition may be formulated to have a weight ratio of the total amount of alpha hydroxyl acid (lactic acid and/or mandelic acid) to total amount of keto acid (e.g. pyruvic acid) of about 10:1 to about 1:1, about 8:1 to about 1:1, about 7:1 to about 1:1, about 6:1 to about 1:1, about 5:1 to about 1:1, about 4:1 to about 1:1; from about 12:1 to about 3:1, about 10:1 to about 3:1, about 8:1 to about 3:1, about 7:1 to about 3:1, about 6:1 to about 3:1, about 5:1 to about 3:1; from about 12:1 to about 4:1, about 10:1 to about 4:1, about 8:1 to about 4:1, about 7:1 to about 4:1, about 6:1 to about 4:1, about 5:1 to about 4:1; from about 12:1 to about 5:1, about 10:1 to about 5:1, about 8:1 to about 5:1, about 7:1 to about 5:1, about 6:1 to about 5:1; from about 12:1 to about 6:1, about 10:1 to about 6:1, about 8:1 to about 6:1, about 7:1 to about 6:1; from about 12:1 to about 7:1, about 10:1 to about 7:1, about 8:1 to about 7:1, or any range or subrange thereof.

The personal care compositions may, in some cases, be free or substantially free of hydroquinone. In some cases, the personal care composition includes hydroquinone in an amount of about 3 wt. % or less, about 2 wt. % or less, about 1 wt. % or less, about 0.5 wt. % or less, about 0.1 wt. % or less, or about 0.05 wt. % or less, based on the total weight of the personal care composition. In at least one embodiment, the personal care composition is free of hydroquinone.

Additionally or alternatively, the personal care composition may have a reduced amount, be substantially free of, or be free of resorcinol and/or a derivative thereof. In some instances, the amount of resorcinol and/or a derivative thereof in the personal care composition is about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less, based on the total weight of the personal care composition. In at least one embodiment, however, the personal care composition includes resorcinol and/or a derivative thereof in an amount of about 0.1 to about 5 wt. %, about 0.1 to about 3 wt. %, about 0.1 to about 1 wt. %, about 1 to about 5 wt. %, about 1 to about 3 wt. %, or any range or subrange thereof, based on the total weight of the personal care composition.

In some cases, the personal care composition may be free of or substantially free of kojic acid. For instance, the personal care composition may include kojic acid in an amount of about 3 wt. % or less, about 2 wt. % or less, about 1 wt. % or less, about 0.5 wt. % or less, about 0.1 wt. % or less, or about 0.05 wt. % or less, based on the total weight of the personal care composition. In at least one embodiment, the personal care composition is free of kojic acid.

Suitable components, such as those listed below, may be included or excluded from the formulations for the personal care compositions depending on the specific combination of other ingredients and the form of the personal care compositions. The personal care compositions may be formulated for hair care, skin care, and/or sun care. In some embodiments, the personal care composition is applied to the person's skin or scalp. Examples of personal care compositions include an antiperspirant, a deodorant, a body wash, a shower gel, a lotion, a bar soap, a soft soap, a shampoo, a hair conditioner, a sunscreen, a peel (e.g., a chemical peel), and a cosmetic. The personal care composition may be classified and regulated by national or international regulatory agencies as a cosmetic, or as a drug.

As mentioned above, the personal care compositions include tranexamic acid and mandelic acid, preferably, in certain weight ratios. The tranexamic acid may be present in the personal care composition in amount from about 0.1 to about 10 wt. %, based on the total weight of the personal care composition. For example, the personal care composition may include tranexamic acid in an amount from about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %; from about 7 to about 10 wt. %, or any range or subrange thereof, based on the total weight of the personal care composition. In at least one embodiment, the amount of tranexamic acid present in the personal care composition may be about 0.7 to about 2 wt. %, about 0.7 to about 1.5, or about 1 wt. %, based on the total weight of the personal care composition.

The mandelic acid may be present in the personal care composition in an amount from about 2 to about 20 wt. %, based on the total weight of the personal care composition. In some cases, the amount of mandelic acid present in the personal care composition is from about 2 to about 20 wt. %, about 2 to about 16 wt. %, about 2 to about 12 wt. %, about 2 to about 10 wt. % about 2 to about 8 wt. %, about 2 to about 6 wt. %; from about 4 to about 20 wt. %, about 4 to about 16 wt. %, about 4 to about 12 wt. %, about 4 to about 10 wt. %, about 4 to about 8 wt. %, about 4 to about 6 wt. %; from about 6 to about 20 wt. %, about 6 to about 16 wt. %, about 6 to about 12 wt. %, about 6 to about 10 wt. %, about 6 to about 8 wt. %; from about 8 to about 20 wt. %, about 8 to about 16 wt. %, about 8 to about 12 wt. %, about 8 to about 10 wt. %; from about 10 to about 20 wt. %, about 10 to about 16 wt. %, about 10 to about 12 wt. %, or any range or subrange thereof, based on the total weight of the personal care composition. In at least one embodiment, the mandelic acid may be present in an amount of about 7 to about 12 wt. %, about 7 to about 10 wt. %, about 7 to about 9 wt. % or about 8 wt. %, based on the total weight of the personal care composition.

The personal care composition, in some embodiments, includes sodium pyruvate and/or an acid thereof (e.g., pyruvic acid). The personal care compositions may include sodium pyruvate and/or an acid thereof in an amount from about 0.5 to about 10 wt. %, based on the total weight of the personal care composition. For instance, the total amount of sodium pyruvate and/or an acid thereof present in the personal care composition may be from about 0.5 to about 10 wt. %, about 0.5 to about 9 wt. %, about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %; from about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %; from about 2 to about 10 wt. %, about 2 to about 9 wt. %, about 2 to about 8 wt. %, about 2 to about 7 wt. %, about 2 to about 6 wt. %, about 2 to about 5 wt. %, about 2 to about 4 wt. %; from about 3 to about 10 wt. %, about 3 to about 9 wt. %, about 3 to about 8 wt. %, about 3 to about 7 wt. %, about 3 to about 6 wt. %; from about 4 to about 10 wt. %, about 4 to about 9 wt. %, about 4 to about 8 wt. %, about 4 to about 7 wt. %, about 4 to about 6 wt. %, including any range or subrange thereof, based on the total weight of the personal care composition.

In some instances, the personal care compositions include one or more additional acid having a carbon group comprising 1 to 6 carbons, e.g., in an amount from about 1 to about 30 wt. %, based on the total weight of the personal care composition. The one or more additional acid having a carbon group comprising 1 to 6 carbons may be selected from hydrocarbyl acids of formula Cr₃H₂ₙ+1COOH, wherein n is 0 to 6, or acids that are substituted with oxygen containing groups such as alcohols or oxo groups.

Examples of additional acids having a carbon group comprising 1 to 6 carbons include formic acid, methanoic acid, HCOOH, acetic acid, mandelic acid; ethanoic acid, CH₃COOH, propionic acid, CH₃CH₂COOH, butyric acid, butanoic acid, CH₃CH₂CH₂COOH, isobutyric acid, 2-methylpropanoic acid, (CH₃)₂CHCOOH, valeric acid, pentanoic acid, CH₃CH₂CH₂CH₂COOH, isovaleric acid, 3-methylbutanoic acid, and (CH₃)₂CHCH₂COOH. In some embodiments, the additional acid(s) having a carbon group comprising 1 to 6 carbons is/are selected from: lactic acid; pyruvic acid; mandelic acid; butanedioic acid (e.g. succinic acid); formic acid; propionic acid; butyric acid; isobutyric acid; valeric acid; isovaleric acid; a derivative thereof; a salt thereof (e.g. sodium pyruvate); and a combination of two or more thereof. In certain embodiments, the one or more additional acid is selected from: lactic acid; pyruvic acid; mandelic acid; succinic acid; a derivative thereof; a salt thereof; and a combination of two or more thereof.

Examples of additional acids comprising one carbon include methanoic acid, formic acid, and HCOOH.

Examples of additional acids comprising two carbons include ethanoic acid, acetic acid, CH₃COOH, trichloro acetic acid, thio acetic acid, ethanedioic acid, oxalic acid, HOOCCOOH, oxoethanoic acid, glyoxylic acid, formylformic acid, OHCCOOH, 2-hydroxyethanoic acid, glycolic acid, dicarbonous acid, hydroxyacetic acid, and HOCH₂COOH.

Examples of additional acids comprising three carbons include propanoic acid, ethanecarboxylic acid, CH₃CH₂COOH, prop-2-enoic acid, acrylic acid, acroleic acid, ethylenecarboxylic acid, propene acid, vinylformic acid, CH₂═CH—COOH, 2-propynoic acid, propiolic acid, acetylene carboxylic acid, propargylic acid, CH≡C—COOH, propanedioic acid, malonic acid, methanedicarboxylic acid, HOOC—CH₂—COOH, 2-hydroxypropanedioic acid, tartronic acid, hydroxymalonic acid, HOOC—CHOH—COOH, oxopropanedioic acid, mesoxalic acid, ketomalonic acid, HOOC—CO—COOH, 2,2-dihydroxypropanedioic acid, dihydroxymalonic acid, mesoxalic acid monohydrate, HOOC—C(OH)₂—COOH, 2-oxopropanoic acid, pyruvic acid, α-ketopropionic acid, acetylformic acid, pyroracemic acid, CH₃—CO—COOH, 2-hydroxypropanoic acid, lactic acid, milk acid, CH₃—CHOH—COOH, 3-hydroxypropanoic acid, hydracrylic acid, CH₂OH—CH₂—COOH, 2,3-dihydroxypropanoic acid, glyceric acid, CH₂OH—CHOH—COOH, 2-oxiranecarboxylic acid, and glycidic acid.

Examples of additional acids comprising four carbons include butanoic acid, butyric acid, propanecarboxylic acid, CH₃(CH₂)₂COOH, 2-methylpropanoic acid, isobutyric acid, isobutanoic acid, (CH₃)₂CHCOOH, 2-oxobutanoic acid, alpha-ketobutyric acid, CH₃—CH₂—CO—COOH, 3-oxobutanoic acid, acetoacetic acid, CH₃CO—CH₂—COOH, 4-oxobutanoic acid, succinic semialdehyde, HC(O)—CH₂—CH₂—COOH, (E)-butenedioic acid, fumaric acid, trans-1,2-ethylenedicarboxylic acid, 2-butenedioic acid, trans-butenedioic acid, allomaleic acid, boletic acid, donitic acid, lichenic acid, HOOC—CH═CH—COOH, (Z)-butenedioic acid, maleic acid, cis-butenedioic acid, maleinic acid, toxilic acid, HOOC—CH═CH—COOH, oxobutanedioic acid, oxaloacetic acid, oxalacetic acid, oxosuccinic acid, HOOC—CH₂—CO—COOH, hydroxybutanedioic acid, malic acid, hydroxybutanedioic acid, HOOC—CH₂—CHOH—COOH, 2,3-dihydroxybutanedioic acid, tartaric acid, 2,3-dihydroxysuccinic acid, threaric acid, racemic acid, uvic acid, paratartaric acid, HOOC(CHOH)₂COOH, (E)-but-2-enoic acid, crotonic acid, trans-2-butenoic acid, beta-methylacrylic acid, 3-methylacrylic acid, (E)-2-butenoic acid, and CH₃—CH═CH—COOH.

Examples of additional acids comprising five carbons include pentanoic acid, valeric acid, valerianic acid, butane-1-carboxylic acid, CH₃(CH₂)₃COOH, 3-methylbutanoic acid, isovaleric acid, (CH₃)₂CH—CH₂—COOH, pentanedioic acid, glutaric acid, propane-1,3-dicarboxylic acid, 1,3-propanedicarboxylic acid, n-pyrotartaric acid, HOOC—(CH₂)₃—COOH, 2-oxopentanedioic acid, alpha-ketoglutaric acid, 2-ketoglutaric acid, α-ketoglutaric acid, 2-oxoglutaric acid, oxoglutaric acid, and HOOC—(CH₂)₂—CO—COOH.

In some embodiments, the one or more additional acid having a carbon group comprising 1 to 6 carbons is selected from lactic acid, pyruvic acid, maleic acid, succinic acid, formic acid, propionic acid, butyric acid, isobutyric acid, 2-methylpropanoic acid, valeric acid, isovaleric acid, pentanoic acid, a derivative thereof, and a combination of two or more thereof. For example, in one embodiment, the one or more additional acid having a carbon group comprising 1 to 6 carbons is selected from lactic acid, pyruvic acid, maleic acid, succinic acid, butyric acid, a derivative thereof, and a combination of two or more thereof. In another embodiment, the one or more additional acid having a carbon group comprising 1 to 6 carbons is selected from lactic acid, pyruvic acid, maleic acid, a derivative thereof, and a combination of two or more thereof. In yet a further embodiment, the personal care composition comprises lactic acid, optionally, in an amount from about 1 to about 30 wt. %, based on the total weight of the personal care composition.

Lactic acid is a carboxylic acid with the formula CH₃—CH(OH)—COOH. Under one embodiment, the lactic acid is L-(+)-lactic acid or (S)-lactic acid. Under another embodiment, the lactic acid is D-(−)-lactic acid of (R)-lactic acid. Under yet another embodiment, the lactic acid is a mixture of the two stereoisomers.

The personal care compositions may include a monoalcohol, such as those having 1 to 8 carbons. The amount of monoalcohol in the personal care composition may be from about 5 to about 50 wt. %, based on the total weight of the personal care composition. For example, the personal care composition may include one or more monoalcohol(s) in an amount from about 5 to about 45 wt. %, about 5 to about 40 wt. %, about 5 to about 38 wt. %, about 5 to about 36 wt. %, about 5 to about 34 wt. %, about 5 to about 32 wt. %, about 5 to about 30 wt. %, about 5 to about 28 wt. %, about 5 to about 26 wt. %; from about 10 to about 50 wt. %, about 10 to about 45 wt. %, about 10 to about 40 wt. %, about 10 to about 38 wt. %, about 10 to about 36 wt. %, about 10 to about 34 wt. %, about 10 to about 32 wt. %, about 10 to about 30 wt. %, about 10 to about 28 wt. %, about 10 to about 26 wt. %; from about 15 to about 50 wt. %, about 15 to about 45 wt. %, about 15 to about 40 wt. %, about 15 to about 38 wt. %, about 15 to about 36 wt. %, about 15 to about 34 wt. %, about 15 to about 32 wt. %, about 15 to about 30 wt. %, about 15 to about 28 wt. %; from about 20 to about 50 wt. %, about 20 to about 45 wt. %, about 20 to about 40 wt. %, about 20 to about 38 wt. %, about 20 to about 36 wt. %, about 20 to about 34 wt. %, about 20 to about 32 wt. %, about 20 to about 30 wt. %; from about 25 to about 50 wt. %, about 25 to about 45 wt. %, about 25 to about 40 wt. %, about 25 to about 38 wt. %, about 25 to about 36 wt. %, about 25 to about 34 wt. %; from about 30 to about 50 wt. %, about 30 to about 45 wt. %, about 30 to about 40 wt. %, about 30 to about 38 wt. %, or any range or subrange thereof, based on the total weight of the personal care composition.

Non-limiting examples of monoalcohols includes methanol, CH₃OH, ethanol, CH₃CH₂OH, n-propanol, 1-propanol, CH₃—CH₂—CH₂—OH, iso-propanol, 2-propanol, (CH₃)₂CH—OH, n-butanol, 1-bunatol, CH₃—CH₂—CH₂—CH₂—OH, sec-butanol, 2-butanol, CH₃—CH₂—CHOH—CH₃, iso-butanol, (CH₃)₂CH—CH₂—OH, tert-butanol, (CH₃)₃C—OH, normal amyl alcohol, pentan-1-ol, CH₃—(CH₂)₄OH, isobutyl carbinol, 3-methylbutan-1-ol, isoamyl alcohol, isopentyl alcohol, (CH₃)₂CH—CH₂—CH₂—OH, active amyl alcohol, 2-methylbutan-1-ol, CH₃—CH₂—C(CH₃)H—CH₂—OH, tertiary butyl carbinol, 2,2-dimethylpropan-1-ol, neopentyl alcohol, (CH₃)₃C—CH₂—OH, 3-pentanol, pentan-3-ol, (CH₃—CH₂)CH—OH, methylpropyl carbinol, pentan-2-ol, CH₃—CH₂—CH₂—CH(OH)—CH₃, methyl isopropyl carbinol, 3-methylbutan-2-ol, (CH₃)₂CH—C(OH)—CH₃, dimethyl ethyl carbinol, 2-methylbutan-2-ol, and tertiary amyl alcohol, CH₃—CH₂—C(CH₃)₂—OH.

Additionally or alternatively, the personal care composition may include one or more polyol(s), e.g., in an amount from about 0.1 to about 20 wt. %, based on the total weight of the personal care composition. For instance, the amount of polyol(s) present in the personal care composition may be from about 0.1 to about 15 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 20 wt. %, about 0.5 to about 15 wt. %, about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 20 wt. %, about 1 to about 15 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 3 to about 20 wt. %, about 3 to about 15 wt. %, about 3 to about 10 wt. %, about 3 to about 8 wt. %, about 3 to about 6 wt. %; from about 5 to about 20 wt. %, about 5 to about 15 wt. %, about 5 to about 10 wt. %, about 5 to about 8 wt. %; from about 7 to about 20 wt. %, about 7 to about 15 wt. %, about 7 to about 10 wt. %; from about 9 to about 20 wt. %, about 9 to about 15 wt. %; from about 11 to about 20 wt. %, about 11 to about 15 wt. %; or about 14 to about 20 wt. %, including any range or subrange thereof, based on the total weight of the personal care composition.

The term “polyol” should be understood as meaning, within the meaning of the present disclosure, an organic molecule comprising at least two free hydroxyl groups. The polyols of the personal care composition may be glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, and dipropylene glycol, and mixtures thereof. In some cases, the polyol is propylene glycol. In some further cases, the polyol is one or both of propylene glycol and butylene glycol. Additionally, in some cases, the personal care composition comprises at least propylene glycol, and optionally one or more polyols other than propylene glycol.

Non-limiting examples of polyols that may, optionally, be included in the personal care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or are chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and a mixture thereof.

The personal care composition may include water, e.g., in an amount of about 49 wt. % or less, based on the total weight of the personal care composition. For example, water may be present in the personal care composition in an amount of from about 40 wt. % or less, about 35 wt. % or less, about 30 wt. % or less, about 25 wt. % or less, about 20 wt. % or less, about 15 wt. % or less, about 10 wt. % or less, about 5 wt. % or less, about 4 wt. % or less, about 3 wt. % or less, about 2 wt. % or less, about 1 wt. % or less, about 0.5 wt. % or less, or about 0.1 wt. % or less. In some cases, the personal care compositions are formulated to have an amount of water from about 1 to about 49 wt. %, about 10 to about 49 wt. %, about 15 to about 49 wt. %, about 20 to about 49 wt. %, about 25 to about 49 wt. %, about 30 to about 49 wt. %, about 35 to about 49 wt. %, about 40 to about 49 wt. %; from about 1 to about 45 wt. %, about 10 to about 45 wt. %, about 15 to about 45 wt. %, about 20 to about 45 wt. %, about 25 to about 45 wt. %, about 30 to about 45 wt. %, about 35 to about 45 wt. %; from about 1 to about 40 wt. %, about 10 to about 40 wt. %, about 15 to about 40 wt. %, about 20 to about 40 wt. %, about 25 to about 40 wt. %, about 30 to about 40 wt. %; from about 1 to about 37 wt. %, about 10 to about 37 wt. %, about 15 to about 37 wt. %, about 20 to about 37 wt. %, about 25 to about 37 wt. %, about 30 to about 37 wt. %; from about 1 to about 34 wt. %, about 10 to about 34 wt. %, about 15 to about 34 wt. %, about 20 to about 34 wt. %, about 25 to about 34 wt. %; from about 1 to about 31 wt. %, about 10 to about 31 wt. %, about 15 to about 31 wt. %, about 20 to about 31 wt. %, about 25 to about 31 wt. %; from about 1 to about 28 wt. %, about 10 to about 28 wt. %, about 15 to about 28 wt. %, about 20 to about 28 wt. %, or any range or subrange thereof, based on the total weight of the personal care composition.

The personal care composition may, optionally, include a skin conditioning agent. The conditioning agent may be a conditioning oil, such as a hydrocarbon oil, polyolefins, a fatty ester, silicones, etc. In one embodiment, from about 0.05% to about 20%, from about 0.08% to about 1.5%, from about 0.1% to about 1%, of at least one organic conditioning oil is included in the personal care composition as a conditioning agent, either alone or in combination with other conditioning agents, such as the silicones. For example, the personal care composition may include one or more skin conditioning agent(s) in an amount from about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %; from about 7 to about 10 wt. %, or any range or subrange thereof, based on the total weight of the personal care composition.

Non-limiting examples of organic conditioning oils for use as conditioning agents in the personal care compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms. Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation, hydrocarbon polymers such as polybutene and polydecene. A hydrocarbon polymer worth noting is polybutene, such as the copolymer of isobutylene and butene. A commercially available material of this type is L-14 polybutene from Amoco Chemical Corporation.

Examples of organic conditioning oils also include liquid polyolefins, such as liquid poly-α-olefins, preferably hydrogenated liquid poly-α-olefins. Polyolefins for use herein are prepared by polymerization of C₄ to about C₁₄ olefenic monomers, e.g., from about C₆ to about C₁₂. Non-limiting examples of olefenic monomers for use in preparing the polyolefin liquids herein include ethylene, propylene, 1-butene, 1-pentene, 1-hexene to 1-hexadecenes, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

Other examples of organic conditioning oils for use as the conditioning agent include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.). Specific examples of fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other examples of fatty esters include mono-carboxylic acid esters of the general formula R′COOR, wherein R′ and R are alkyl or alkenyl radicals, and the sum of carbon atoms in R′ and R is at least 10, preferably at least 22. Still other fatty esters include di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, and tristearyl citrate.

Other fatty esters suitable include those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol di stearate, polyoxyethylene polyol fatty acid ester, sorbitan fatty acid esters, and polyoxyethylene sorbitan fatty acid esters.

Still other examples of fatty esters include glycerides, including, but not limited to, mono-, di-, and tri-glycerides, preferably di- and tri-glycerides, more preferably triglycerides. The glycerides may be mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Additional fatty esters suitable for use in the compositions of the present invention are water insoluble synthetic fatty esters. Specific non-limiting examples of suitable synthetic fatty esters for use in the compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company. In an embodiment, the composition contains ingredients known as emollients and skin feel modifiers. A wide range of skin feel modifiers can be used, such as Glyceryl Esters and Derivatives (Excluding fats and oils); Alkoxylated Alcohols (Limited to alkoxylation products of alcohols); Ethers (Excluding alkoxylated derivatives). In an embodiment, the skin feel modifiers are selected from polyglyceryl-3 laurate, PPG-3 isostearyl methyl ether, and PEG-60 almond glycerides.

The personal care composition may also include one or more additional ingredients. The CTFA Cosmetic Ingredient Handbook, Tenth Edition (published by the Cosmetic, Toiletry, and Fragrance Association, Inc., Washington, D.C.) (2004) describes a non-limited wide variety of cosmetic and pharmaceutical ingredients usually used in the skin care industry that can be used as additional ingredients in the compositions of the present invention. Examples of these ingredient classes include, but are not limited to: healing agents, skin anti-aging agents, skin moisturizing agents, anti-wrinkle agents, anti-atrophy agents, skin smoothing agents, antibacterial agents, antifungal agents, pesticides anti parasitic agents, antimicrobial agents, anti-inflammatory agents, anti-pruriginous agents, external anesthetic agents, antiviral agents, keratolytic agents, free radicals scavengers, antiseborrheic agents, antidandruff agents, the agents modulating the differentiation, proliferation or pigmentation of the skin and agents accelerating penetration, desquamating agents, depigmenting or propigmenting agents, antiglycation agents, tightening agents, agents stimulating the synthesis of dermal or epidermal macromolecules and/or preventing their degradation; agents stimulating the proliferation of fibroblasts and/or keratinocytes or stimulating the differentiation of keratinocytes; muscle relaxants; antipollution and/or anti-free radical agents; slimming agents, anticellulite agents, agents acting on the microcirculation; agents acting on the energy metabolism of the cells; cleaning agents, hair conditioning agents, hair styling agents, hair growth promoters, sunscreen and/or sunblock compounds, make-up agents, detergents, pharmaceutical drugs, emulsifiers, emollients, antiseptic agents, deodorant actives, dermatologically acceptable carriers, surfactants, abrasives, absorbents, aesthetic components such as fragrances, colorings/colorants, essential oils, skin sensates, cosmetic astringents, anti-acne agents, anti-caking agents, anti-foaming agents, antioxidants, binders, biological additives, enzymes, enzymatic inhibitors, enzyme-inducing agents, coenzymes, plant extracts, plant derivatives, plant tissue extracts, plant seed extracts, plant oils, botanicals, botanical extracts, ceramides, peptides, buffering agents, bulking agents, chelating agents, chemical additives, colorants, cosmetic biocides, denaturants, drug astringents, external analgesics, film formers or materials, e.g., polymers, for aiding the film-forming properties, quaternary derivatives, opacifying agents, pH adjusters, propellants, reducing agents, sequestrants, skin bleaching and lightening agents, skin tanning agents, skin-conditioning agents (e.g., humectants, including miscellaneous and occlusive), skin soothing and/or healing agents and derivatives, skin treating agents, thickeners, and vitamins and derivatives thereof, peeling agents, moisturizing agents, curative agents, lignans, preservatives, UV absorbers, a cytotoxic, an antineoplastic agent, a fat-soluble active, suspending agents, viscosity modifiers, dyes, nonvolatile solvents, diluents, pearlescent aids, foam boosters, a vaccine, and their mixture.

In some embodiments, said additional ingredient is selected from the group consisting of sugar amines, glucosamine, D-glucosamine, N-acetyl glucosamine, N-acetyl-D-glucosamine, mannosamine, N-acetyl mannosamine, galactosamine, N-acetyl galactosamine, vitamin B3 and its derivatives, niacinamide, sodium dehydroacetate, dehydroacetic acid and its salts, phytosterols, salicylic acid compounds, hexamidines, dialkanoyl hydroxyproline compounds, soy extracts and derivatives, equol, isoflavones, flavonoids, phytantriol, farnesol, geraniol, peptides and their derivatives, di-, tri-, tetra-, penta-, and hexapeptides and their derivatives, lys-thr-thr-lys-ser, palmitoyl-lys-thr-thr-lys-ser, carnosine, N-acyl amino acid compounds, retinoids, retinyl propionate, retinol, retinyl palmitate, retinyl acetate, retinal, retinoic acid, water-soluble vitamins, ascorbates, vitamin C, ascorbic acid, ascorbyl glucoside, ascorbyl palmitate, magnesium ascorbyl phosphate, sodium ascorbyl phosphate, vitamins their salts and derivatives, provitamins and their salts and derivatives, ethyl panthenol, vitamin B, vitamin B derivatives, vitamin B1, vitamin B2, vitamin B6, vitamin B12, vitamin K, vitamin K derivatives, pantothenic acid and its derivatives, pantothenyl ethyl ether, panthenol and its derivatives, dexpanthenol, biotin, amino acids and their salts and derivatives, water soluble amino acids, asparagine, alanine, indole, glutamic acid, water insoluble vitamins, vitamin A, vitamin E, vitamin F, vitamin D, mono-, di-, and tri-terpenoids, beta-ionol, cedrol, and their derivatives, water insoluble amino acids, tyrosine, tryptamine, butylated hydroxytoluene, butylated hydroxyanisole, allantoin, tocopherol nicotinate, tocopherol, tocopherol esters, palmitoyl-gly-his-lys, phytosterol, hydroxy acids, glycolic acid, lactic acid, lactobionic acid, keto acids, pyruvic acid, phytic acid, lysophosphatidic acid, stilbenes, cinnamates, resveratrol, kinetin, zeatin, dimethylaminoethanol, natural peptides, soy peptides, salts of sugar acids, Mn gluconate, Zn gluconate, particulate materials, pigment materials, natural colors, piroctone olamine, 3,4,4′-trichlorocarbanilide, triclocarban, zinc pyrithione, hydroquinone, kojic acid, ascorbic acid, magnesium ascorbyl phosphate, ascorbyl glucoside, pyridoxine, aloe vera, terpene alcohols, allantoin, bisabolol, dipotassium glycyrrhizinate, glycerol acid, sorbitol acid, pentaerythritol acid, pyrrolidone acid and its salts, dihydroxyacetone, erythrulose, glyceraldehyde, tartaraldehyde, clove oil, menthol, camphor, eucalyptus oil, eugenol, menthyl lactate, witch hazel distillate, eicosene and vinyl pyrrolidone copolymers, iodopropyl butylcarbamate, a polysaccharide, an essential fatty acid, salicylate, glycyrrhetinic acid, carotenoides, ceramides and pseudo-ceramides, a lipid complex, oils in general of natural origin such as shea butter, shea oil, apricot oil, onagre oil, prunus oil, palm oil, monoi oil, HEPES; procysteine; O-octanoyl-6-D-maltose; the disodium salt of methylglycinediacetic acid, steroids such as diosgenin and derivatives of DHEA; DHEA or dehydroepiandrosterone and/or a precursor or chemical or biological derivative, N-ethyloxycarbonyl-4-para-aminophenol, bilberry extracts; phytohormones; extracts of the yeast Saccharomyces cerevisiae; extracts of algae; extracts of soyabean, lupin, maize and/or pea; alverine and its salts, in particular alverine citrate, extract of butcher's broom and of horse chestnut, metallopreoteinase inhibitor, and combinations of two or more thereof.

The personal care composition may include a preservative. The preservative(s) may be selected from benzalkonium chloride; benzethonium chloride, 5-bromo-5-nitro-1,3-dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynl butyl carbamate, butyl paraben; ethyl paraben; methyl paraben; propyl paraben, mixture of methyl isothiazolinone/methyl-chloroisothiazolinone in a 1:3 wt. ratio; mixture of phenoxyethanol/butyl paraben/methyl paraben/propylparaben; 2-phenoxyethanol; tri s-hydroxyethyl-hexahydrotriazine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadamantane chloride; organic acids, lactic acid, or citric acid, and combinations of two or more thereof. In an embodiment, the preservative comprises sorbitan caprylate, propanediol, and benzoic acid, available commercially as Nipaguard SCE. In embodiments, the one or more preservative(s) is present in an amount from about 0.25 wt. % to 3 wt. %, or 0.3 wt. % to 2 wt. %, or 0.3 wt. % to 0.7 wt. %, based on the total weight of the personal care composition.

In accordance with another aspect of the invention, provide is a method for improving the health and/or appearance of skin. The method typically comprises applying a personal care composition as disclosed herein, preferably, in an effective amount. The methods of the present disclosure may include applying to the skin of an individual a personal care composition topically, subcutaneously, or transdermally. Subcutaneous administration may utilize injection, or may proceed using a patch, poultice, microneedle, or iontophoresis. Examples of topical administration include by way of application of the personal care composition in the form of a cream, a lotion, a serum, an extract, or in a layer (e.g., a gel layer) of a patch.

In some embodiments, the method comprises wounding a section of skin and applying a personal care composition disclosed herein to the wounded section of skin. The personal care composition may be applied before or after wounding of the skin. The section of skin may be wounded by way of microneedling, e.g., using a microneedling device and procedures known by one of ordinary skill in the art. For example, the method may comprise applying the personal care composition to a section of skin of the individual and subsequently microneedling the section of skin to deliver the personal care composition or component thereof into or below the stratum corneum. The method may use the microneedle device to pierce the stratum corneum substantially without penetrating the dermis to target the tissue layers below the stratum corneum.

The microneedle device employed by the method may include a plurality of microneedles, which may have a length sufficient to penetrate the stratum corneum. The microneedles may be hollow and/or have central passages extending through the lengths of the microneedles for communicating with a syringe barrel to enable pressurized application of the personal care composition through the stratum corneum of the skin. In some embodiments, however, the microneedles are solid.

In at least one embodiment, the method is iontophoresis and/or includes applying an electrical current to the skin. The application of microneedles can decrease the electrical resistance of the stratum corneum, and so the voltage necessary to facilitate iontophoresis can be greatly reduced, improving safety and requiring much less power consumption. By use of the iontophoresis, certain compounds of the personal care may travel through the stratum corneum and into the thicker dermis, and the combination of both transport-enhancing methods provides an increase in permeability for both the stratum corneum and the deeper layers of the skin. In some instances, the transport improvement in the stratum corneum is mostly due to microneedle piercing, while the iontophoresis provides higher transport rates in the epidermis and dermis. Microneedle devices and methods of use thereof may be found in U.S. Pat. No. 6,623,457, which is incorporated herein by reference in its entirety.

### EXAMPLES

### Example 1

A non-limiting, exemplary personal care composition (Example Composition A) was prepared in accordance with aspects of the invention. A comparative composition (Comparative Composition 1) containing kojic acid and hydroquinone and a positive control composition (Comparative Composition 2) containing kojic acid were also prepared. The formulations for Example Composition A and Comparative Compositions 1 and 2 are summarized in Table 1, provided below.

TABLE 1

| US INCI Compound | Ex. A | Comp. 1 | Comp. 2 |
|---|---|---|---|
| Name | (wt. %) | (wt. %) | (wt. %) |
| Tranexamic Acid | 0.5-2 | | |
| Mandelic Acid | 7-12 | | |
| Lactic Acid | 16 | 15.7 | 15.7 |
| Sodium Pyruvate | 4 | | |
| Salicylic Acid | | 14.4 | 14.4 |
| Kojic Acid | | 3.1 | 3.1 |
| Hydroquinone | | 2 | |
| Ethanol and/or Isopropyl | 29.6 | 52.5 | 52.5 |
| Alcohol | | | |
| Polysorbate 20 | 2 | | |
| Caprylyl Glycol | 1 | | |
| Leuconostoc/Radish Root | 1.5 | | |
| Ferment Filtrate and | | | |
| Silybum Marianum (Milk | | | |
| Thistle) Extract | | | |
| Sodium Gluconate | 0.2 | | |
| Bisabolol (L-alpha), | 0.15 | | |
| Glycyrrhetinic Acid, and | | | |
| Thioctic (R-lipoic) Acid | | | |
| Citric Acid | <0.05 | 10.3 | 10.3 |
| Sodium Hydroxide | 0.01 | <0.1 | <0.1 |
| Water | 36.5 | 4.2 | 2.2 |

### Example 2

Example Composition A was evaluated in comparison to Comparative Composition 1 to assess the effect on melanin pigmentation using skin explants. The skin explants had a skin type IV and were obtained from a 40 year old female donor. Samples from each of Example Composition A and Comparative Composition 1 were applied to respective skin explants and left on the respective skin explains for 20 minutes. The explants were then harvested on day 7 for evaluation.

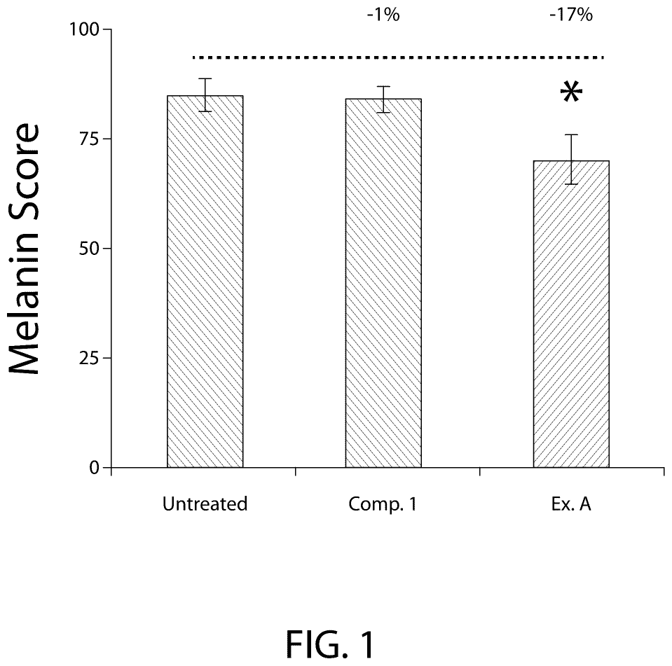

The skin explants were evaluated by way of histochemical imaging to determine the level of melanin pigmentation in the skin explants. Specifically, the skin explants were stained with Fontana Masson (melanin) and picosirius red (collagen), imaged with bright field microscopy, and then transformed from RGB images into L*a*b* color scale to quantify melanin and collagen content. As seen in FIG. 1, Example Composition A achieved a significant reduction in melanin as compared to Comparative Composition 1.

### Example 3

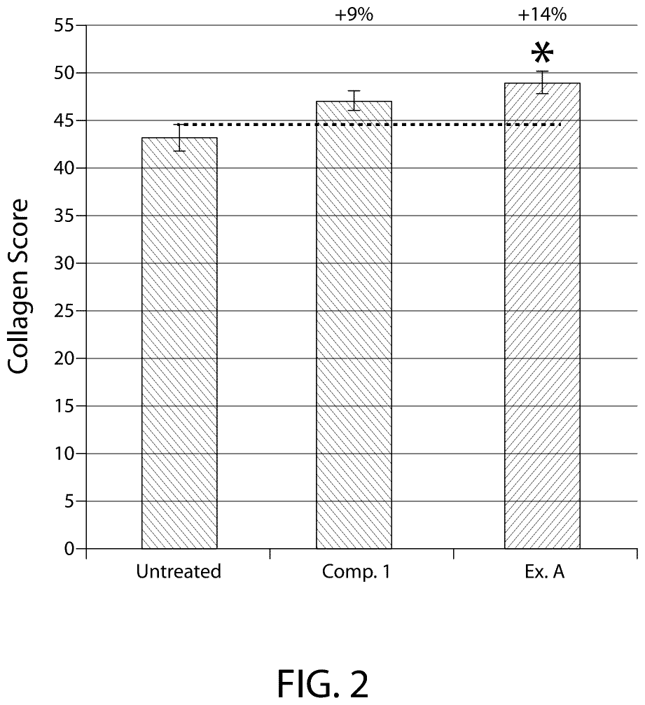

A study was conducted to evaluate the effect of Example Composition A and Comparative Composition 1 on the collagen within skin. Specifically, samples of Example Composition A and Comparative Composition 1 were applied to skin explants according to the procedures described in Example 1, except that the stain for imaging the skin explants was specific to collagen imaging. Example Composition A unexpectedly provided an improvement in collagen growth as seen in FIG. 2.

### Example 4

Example Composition A was evaluated to assess the three markers of pigmentation (TYR, TYRP1, and DCT) in comparison to Comparative Composition 1 and Comparative Composition 2. Samples from Example Composition A and Comparative Composition 1 were applied to respective skin explants. The skin explants had a skin type II and were obtained from a 33 year old female donor. Similar to Example 1, the samples of Example Composition A and Comparative Composition 1 applied to the skin explants for 20 minutes. The skin explants were then harvested and evaluated 7 days after the administration of the respective sample compositions.

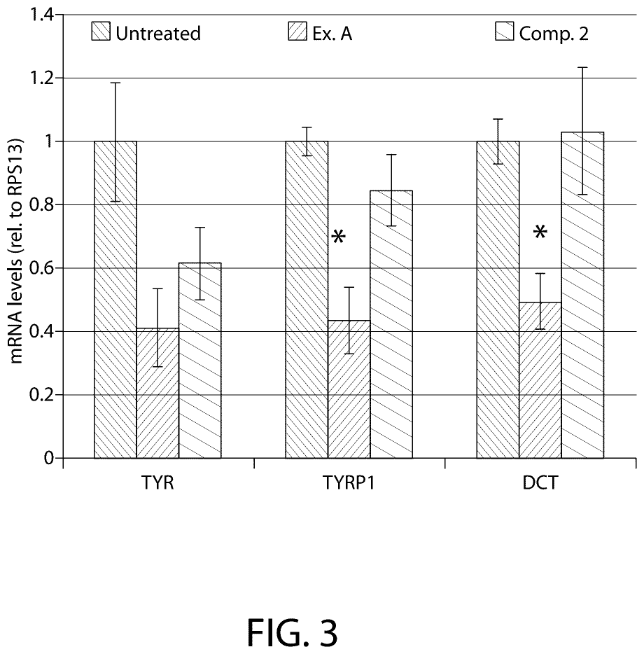

The skin explants were then evaluated to determine the level of three markers of pigmentation, namely TYR, TYRP1, and DCT, in the skin explants. The skin explants were then evaluated using procedures similar to those described in Example 2, except the difference of analytical method, RT-qPCR to quantify gene expression. The level of the three gene markers were then normalized to housekeeping gene Ribosomal protein S13. Notably, Example Composition A significantly reduced the level of TYRP1, and DCT as compared to Comparative Compositions 1 and 2. FIG. 3 is a bar graph of the mRNA levels for TYR, TYRP1, and DCT in skin explants seven days after a single application of one of two comparative compositions or a non-limiting, exemplary composition according to aspects of the invention.

### Example 5

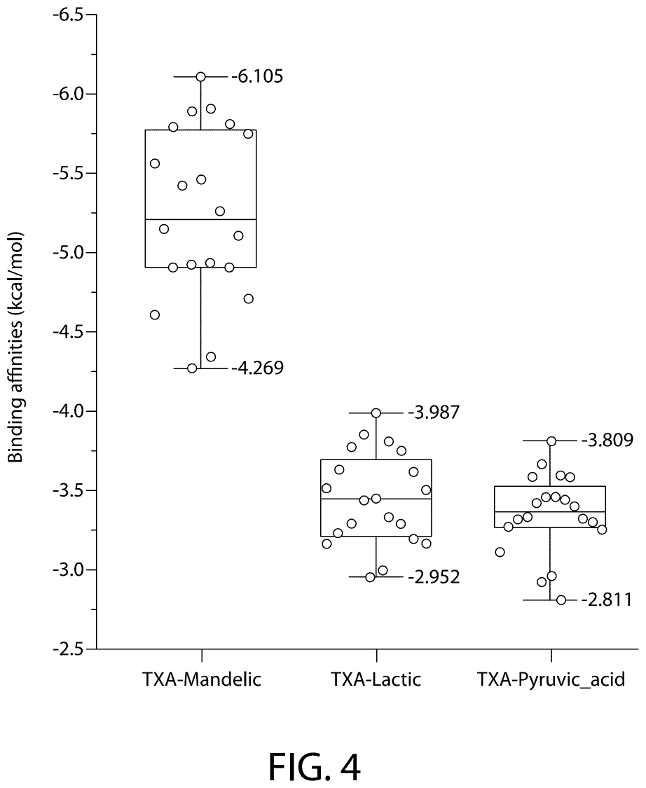

Three compositions were prepared to evaluate the binding affinity of tranexamic acid (“TXA”) in combination with mandelic acid, lactic acid, or pyruvic acid. Each of the compositions was prepared with the same amount of tranexamic acid and had molar equivalent amounts of mandelic acid, lactic acid, or pyruvic acid relative to one another. As seen in FIG. 4, the composition containing tranexamic acid and mandelic acid had significantly higher binding affinities than the composition containing tranexamic acid and latic acid and the composition containing tranexamic acid and pyruvic acid.

### Example 6

Example Composition A was further evaluated to assess the expression of melanin synthesis gene markers. Specifically, 9 consenting volunteers representing different ethnicities, fitzpatrick skin types (I-V) and skin conditions (photodamage, melasma and acne-induced post-inflammatory hyperpigmentation) were received 2 peeling sessions, 4 weeks apart. A summary of the application and evaluation of Example Composition A is provided in Table 2 (below).

TABLE 2

| | Intervention/Study Week | | | |
|---|---|---|---|---|
| Procedure | Screening | 0 wk | 4 wks | 8 wks |
| Informed Consent | X | | | |
| Subject self-assessment | | X | X | X |
| Facial swabbing | | X | X | X |
| Visual assessment by | | X | X | X |
| Dermatologist | | | | |
| VISIA-CR imaging | | X | X | X |
| Vivascope Imaging | | X | X | X |
| MPTflex Imaging | | X | X | X |
| Subject self-assessment | | X | X | |
| ( post-toner application) | | | | |
| Chemical-peeling | | X | X | |

There were three measurement sessions: before peel (Week 0), 4 weeks after 1st peel (Week 4) and 4 weeks after 2nd peel (Week 8). Visia-CR 4.3.1 with special optics and RBX image processing to separate red from brown was used to capture and analyze images of the face from three angles (front, left and right). A certified dermatologist assessed the changes in the global appearance of volunteer's face, specifically for skin texture, uneven pigmentation, and redness on the scale of 0-4 (0=No, 1=minimal, 2=mild, 3=moderate, 4=severe).

Optical biopsies of the facial skin were taken using Vivascope 1500 and MPTflex, as reported previously. Specifically, medical grade ECG rings were used to monitor the same spot over the course of treatment (longitudinal imaging). Images of skin anatomy (keratinocytes, melanin, collagen, etc.) were acquired following manufacturer's protocol, using a tissue cap and conducting gel for vivascope 1500 imaging (830 nm laser with maximum power 20 mW) and magnetic ring with coverslip, oil and water for MPTflex imaging (tunable laser at 760 nm and 890 nm with maximum power 39 mW). Dermoscope images were also acquired prior to imaging using vivascope 1500. Vivascope images were studied primarily to visualize melanin, while MPTflex images were used to visualize keratinocytes. The FLIM image and photon counts were acquired at two different wavelengths (760 nm and 890 nm) to accurately discriminate and quantitate the NADH from other autofluorophores in the skin like melanin and FAD. FLIM images were processed offline using SPCImage software from Becker and Hickl GmbH.

Swabbing of the cheeks was done using the special large contour FLOQSwabs® from Copan Diagnostics, Inc. USA (Cat #528C). In particular, the entire cheek was swabbed using a sterile swab after impregnating the applicator of the swab in PBS buffer solution. The cheek was swabbed for 30 seconds by daubing in a rolling manner and in horizontal directions to ensure that the entire surface area of the cheek is covered to accurately represent the sample. After sample collection, the tip of the swab was cut off and placed in a sterile tube. The swabs were stored immediately at a temperature of −80° C. until tested for microbiological information. 16SrRNA sequencing was done using MiSeq device (Illumina) and the data was processed to obtain alpha and beta diversity index. Further analysis was done on the top two most abundant species, P. acne and S. Epidermidis to quantitate changes before and after peeling.

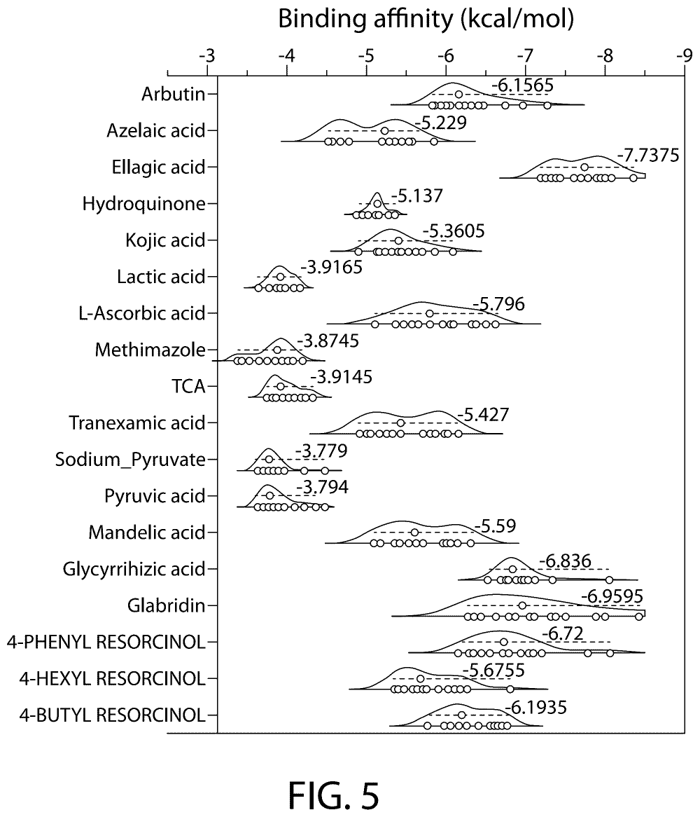

Molecular docking modeling was used to predict the affinity or inhibition of tyrosinase of certain compounds. Specifically, ellagic acid, resorcinol derivatives (Butyl>Phenyl Ethyl), glabridin and glycyrrhizic acid (found commonly in Licorice) and arbutin (a derivative of HQ) exhibited the greatest binding affinity from our in-silico screening, with very high binding affinities of less than −6 kcal/mol (See FIG. 5). Hexyl resorcinol, kojic acid (KA), AzA, HQ, TXA, ascorbic acid, and mandelic acid exhibited a moderate activity (affinity between −4 and −6 kcal/mol), while methimazole, TCA, lactic acid, and pyruvic acid exhibiting the least binding affinity, with binding activities of weaker than −4 kcal/mol.

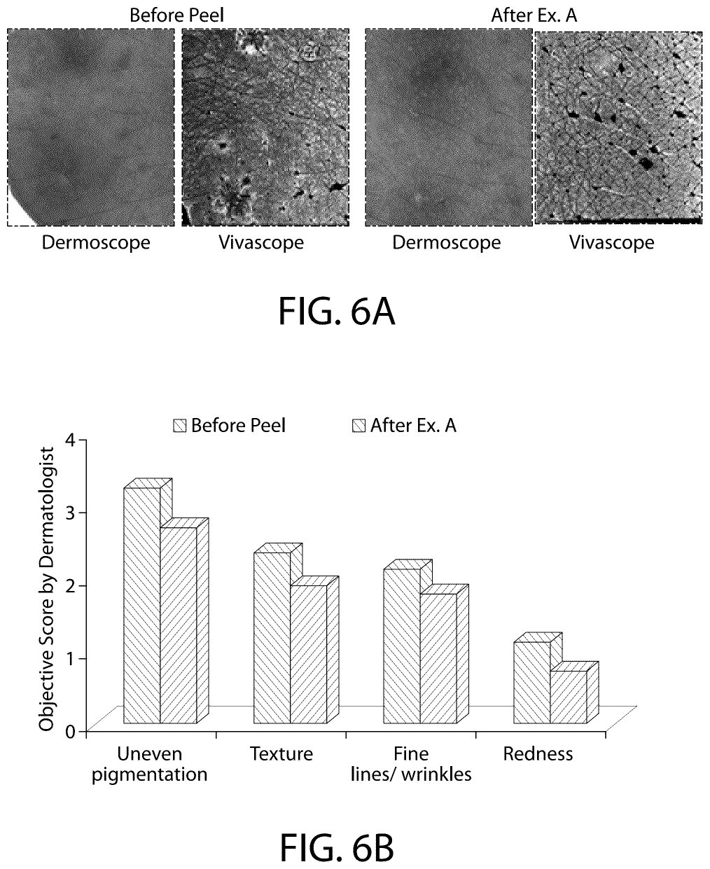

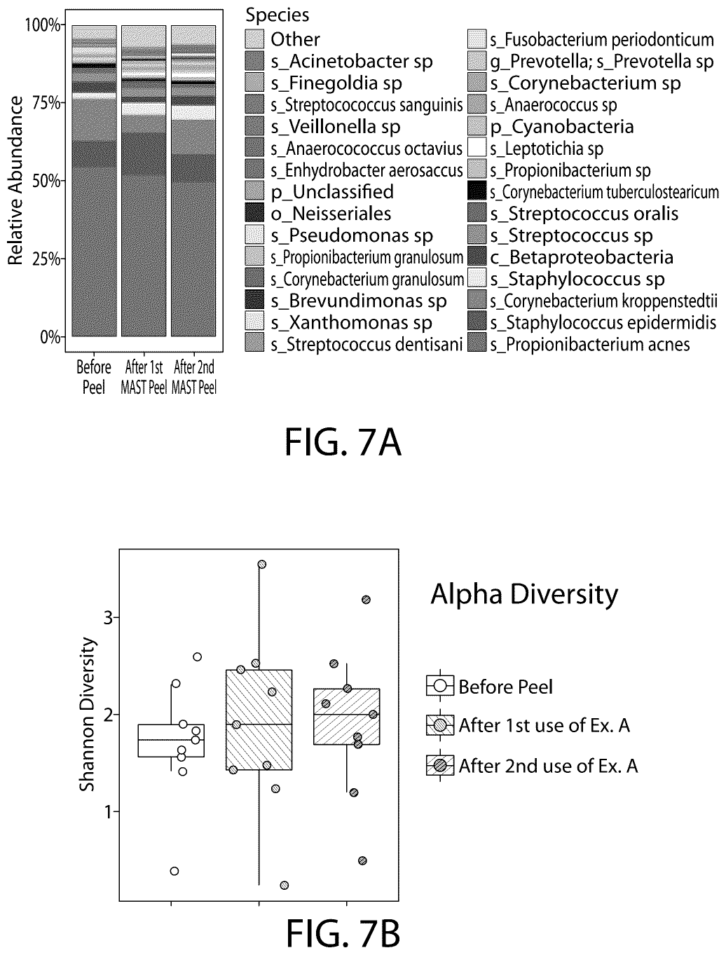

All subjects (N=9) completed the study with no adverse events. A clear decrease in brown patches and redness was observed in most cases where Example Composition A was applied. A decrease in redness and brown spots was even more appreciated after processing of VISIA images using RBX image processing to separate brown from red. The acne-induced PIH case reported a reaction to sunscreen and was corrected after replacing the sunscreen with a commercial sunscreen specially designed for oily and acne-prone skin. FIG. 6A is an image of dermoscope and vivascope images revealing a reduction of acne-induced PIH spots and the complete closure of three comedones after peeling due to application of Example Composition A. FIG. 6B is a bar graph of the average score calculated from the dermatologist's objective visual assessment of each subject before and after chemical peeling with Example Composition A.

Additionally, it was determined that Example Composition A provided a decrease in hyperpigmentation and surprisingly also in erythema in most of the cases we recruited in our study to represent different types of common hyperpigmentation. Based on this surprising determination, it is expected that Example Composition A may be safe for long term use, unlike compositions containing HQ and KA which have risks associated with their long term use.

### Example 7

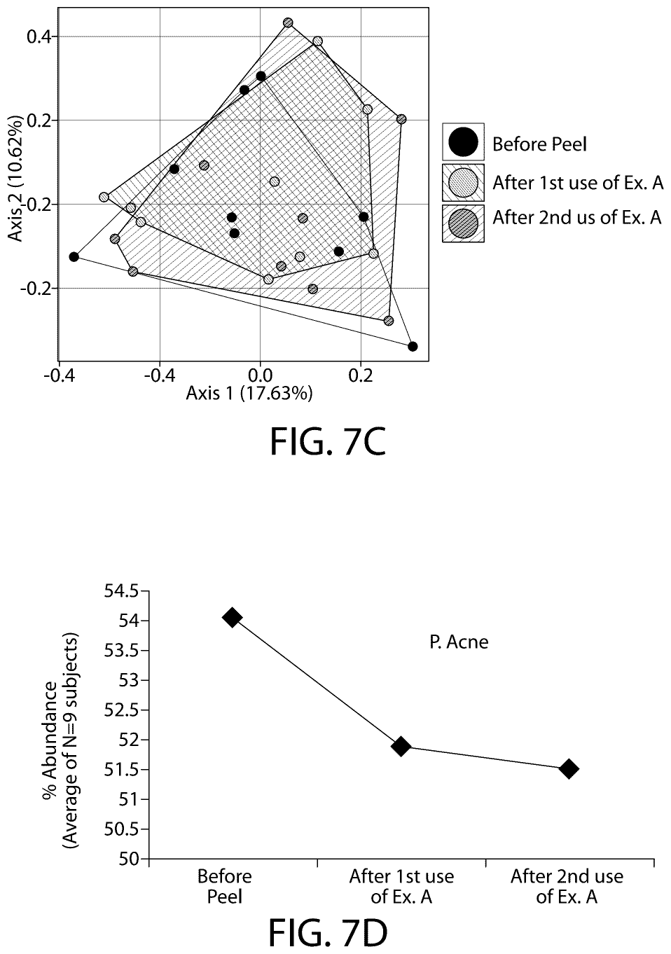

The cheek swab samples from Example 6 were evaluated to assess the microbiome of the volunteers skin after application of Example Composition A. Specifically, from the cheek swab samples (9subjects×3timepoints=27), more than 10,000 reads were produced after gene sequencing, except that one sample that produced 7342 reads. FIG. 7A shows the average microbial composition of the evaluated facial microbiome. P. acnes was most abundant with a range 14-91%, and S. epidermidis was 0.5-26%. S. aureus, typically related with infection and atopic dermatitis was very low, below the detection limit except two subjects with less than 0.1% abundance. Although not significantly different from baseline, there was a noticeable change in species richness, after chemical peeling (FIG. 7B). There was no structural difference (beta diversity) in the microbiome before and after peeling (FIG. 7C). Detailed analysis of all 9 subjects for two most abundant species revealed a noticeable increase in S. epidermidis and a decrease in P. acne, as shown in FIG. 7D. Surprisingly, one more subject (Fitz skin type IV, Hispanic female with severe melasma patches) showed abnormal abundance of P. acne >90%, and a noticeable decrease in porphyrins was observed after peeling with Example Composition A.

### Example 8

Four compositions were prepared to evaluate the effect of the combination of mandelic acid, lactic acid, and pyruvic acid in comparison to the effect of each of the foregoing acids individually. Specifically, a first neat solution was prepared containing lactic acid, a second neat solution was prepared containing mandelic acid, a third neat solution was prepared containing pyruvic acid and a fourth neat solution was prepared containing the combination of lactic acid, mandelic acid, and pyruvic acid in a weight ratio of lactic acid to mandelic acid to pyruvic acid of 4:2:1 and in the same amounts as the respective first, second, and third neat solutions.

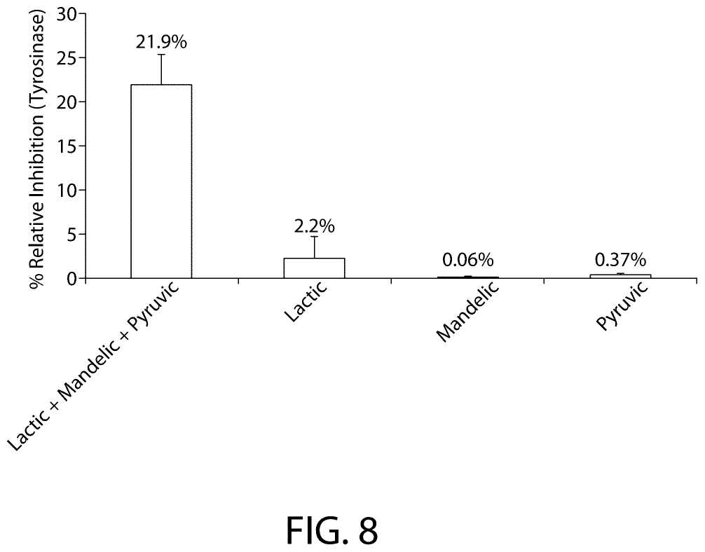

The combination of lactic acid, mandelic acid, and pyruvic acid unexpectedly exhibited a synergistic effect. Specifically, the effect achieved by the combination of mandelic acid, lactic acid, and pyruvic acid was determined to be about 21.9%, which is about tenfold (1000%) higher than the individual additive effect (sum of effect from three individual acids in their respective amounts). FIG. 8 is a bar graph showing the synergistic effect achieved by the combination of mandelic acid, lactic acid, and pyruvic acid. The synergistic effect was particularly surprising in view of the binding activities described in Example 6.

1. A personal care composition comprising:
tranexamic acid; and
mandelic acid,
wherein the personal care composition has a weight ratio of tranexamic acid to mandelic acid of from about 1:6 to about 1:17.

2. The personal care composition according to claim 1, wherein the weight ratio of tranexamic acid to mandelic acid is from about 1:7 to about 1:12.

3. The personal care composition according to claim 1, wherein the tranexamic acid is present in an amount from about 0.1 to about 10 wt. %, based on the total weight of the personal care composition.

4. The personal care composition according to claim 1, wherein the mandelic acid is present in an amount of about 2 to about 20 wt. %, based on the total weight of the personal care composition.

5. The personal care composition according to claim 1 further comprising sodium pyruvate, optionally, in an amount from about 0.5 to about 10 wt. %, based on the total weight of the personal care composition.

6. The personal care composition according to claim 1 further comprising one or more acid having a carbon group comprising 1 to 6 carbons, optionally, in an amount from about 1 to about 30 wt. %, based on the total weight of the personal care composition.

7. The personal care composition according to claim 6, wherein the one or more acid having a carbon group comprising 1 to 6 carbons is selected from lactic acid, pyruvic acid, maleic acid, succinic acid, formic acid, propionic acid, butyric acid, isobutyric acid, 2-methylpropanoic acid, valeric acid, isovaleric acid, pentanoic acid, a derivative thereof, and a combination of two or more thereof.

8. The personal care composition according to claim 6, wherein the one or more acid having a carbon group comprising 1 to 6 carbons is selected from lactic acid, pyruvic acid, maleic acid, succinic acid, butyric acid, a derivative thereof, and a combination of two or more thereof.

9. The personal care composition according to claim 6, wherein the one or more acid having a carbon group comprising 1 to 6 carbons is selected from lactic acid, pyruvic acid, maleic acid, a derivative thereof, and a combination of two or more thereof.

10. The personal care composition according to claim 1 further comprising lactic acid, optionally, in an amount from about 1 to about 30 wt. %, based on the total weight of the personal care composition.

11. The personal care composition according to claim 1, wherein the personal care composition comprises less than about 0.5 wt. % of resorcinol and/or a derivative thereof, based on the total weight of the personal care composition.

12. The personal care composition according to claim 1, wherein the personal care composition is free of resorcinol and/or a derivative thereof.

13. The personal care composition according to claim 1, wherein the personal care composition comprises less than about 1 wt. % of hydroquinone, based on the total weight of the personal care composition.

14. The personal care composition according to claim 1, wherein the personal care composition is free of hydroquinone.

15. The personal care composition according to claim 1 further comprising a monoalcohol in an amount from about 5 to about 50 wt. %, based on the total weight of the personal care composition.

16. The personal care composition according to claim 1 comprising a polyol in an amount from about 0.1 to about 20 wt. %, based on the total weight of the personal care composition.

17. The personal care composition according claim 1, wherein the personal care composition comprises less than about 1 wt. % of kojic acid, based on the total weight of the personal care composition.

18. A personal care composition comprising:
from about 0.1 to about 10 wt. % of tranexamic acid;
from about 2 to about 20 wt. % of mandelic acid;
from about 0.5 to about 10 wt. % of sodium pyruvate; and
optionally, from about 1 to about 30 wt. % of one or more additional acid selected from lactic acid, maleic acid, succinic acid, formic acid, propionic acid, butyric acid, isobutyric acid, 2-methylpropanoic acid, valeric acid, a derivative thereof, and a combination of two or more thereof,
wherein the personal care composition has a weight ratio of tranexamic acid to mandelic acid of from about 1:6 to about 1:17, and all weight percentages are based on the total weight of the personal care composition.

19-34. (canceled)

35. A method for treating, improving, inhibiting or preventing:
dyspigmentation of the skin;
wrinkles;
acne or rosacea;
loss of skin elasticity;
crows feet;
photodamage;
fine lines and wrinkles;
skin tone unevenness;
mottled pigmentation;
pigmented lesions (freckles, sun spots, melasma); and/or
photo damage to the skin,
comprising:
applying a personal care composition according to claim 1, to a skin surface of a subject in need thereof.

36. The method according to claim 35, wherein the personal care composition is applied to the skin surface after a dermatological procedure, wherein the dermatological procedure is selected from: microdermabrasion; ablative laser resurfacing; non-ablative resurfacing; microneedling; ultrasound; cryotherapy; radiofrequency; and a combination of two or more thereof.
